(19) United States
(12) Patent Application Publication
(10) Pub. No.: US 2024/0081803 A1
(43) Pub. Date: Mar. 14, 2024

(54) CLOSED SUTURE CONSTRUCTS AND METHODS FOR TISSUE REPAIR

(71) Applicant: RIVERPOINT MEDICAL, LLC, Portland, OR (US)

(72) Inventors: Elliot BIXBY, Milwaukie, OR (US); Nathan COOK, Portland, OR (US)

(21) Appl. No.: 18/243,450

(22) Filed: Sep. 7, 2023

Related U.S. Application Data
(60) Provisional application No. 63/405,426, filed on Sep. 10, 2022.

(51) Int. Cl. A61B 17/04; A61B 17/06

(52) U.S. Cl. CPC A61B 17/0401; A61B 17/06166; A61B 2017/00831; A61B 2017/0404; A61B 2017/06185

(57) ABSTRACT

Closed Suture Anchor construct and/or assembly having one or more anchoring constructs enclosed by a continuous (i.e., closed) loop of a suture or suture, such as ultra high molecular weight polyethylene (UHMWPE) are disclosed herein. Preferred assemblies are constructed such that the anchoring construct cannot be detached from the continuous loop without breaking or opening the continuous or closed loop of the closed suture anchor construct.

### CROSS REFERENCE TO RELATED APPLICATIONS

This application claims the benefit of U.S. Provisional Application No. 63/405,426 entitled “Constructs and Methods for Tissue Repair,” filed Sep. 10, 2022, the disclosure of which are incorporated by reference herein in its entirety.

### FIELD OF THE INVENTION

The apparatus and methods relate to surgical repair to approximate or repair tears in soft tissue. More specifically, the apparatus and methods relate to surgical repair of meniscal tissue using a suture anchor construct or assembly.

### BACKGROUND OF THE INVENTION

Tissue repair requires the surgeon to pass suture material through selected tissue, form a plurality of surgical knots extracorporeally and then move the knots into position adjacent the desired tissue to be sutured. In such procedures, the surgeon must manually tie the knots on the suture strands after the suture is threaded through the selected tissues to be sutured. Tying knots is well worth avoiding because not only is the task time consuming, but also it creates a potential failure point.

Accordingly, there is a need for suture constructs or assemblies that are both easy-to-use and that relieve a surgeon from the task of tying a knot, and improves the repairing of tears in soft tissue that do not require multiple knots.

### BRIEF SUMMARY OF THE INVENTION

In one embodiment, a “closed” knotless suture tissue connecting construct comprises: a finger trap region, the finger trap region including a first end and a second end; a fixed loop, the fixed loop extending outwardly from the first end of the finger trap region, the fixed loop having a locking point; and a tucked tail, the tucked tail passes through the fixed loop and is inserted into the second end of the finger trap region to exit out of the first end of the finger trap region to create one or more sliding loops. The tucked tail is movable relative to the finger trap region from a first position to a second position, the second position extending distally from the first end of the finger trap region. The finger trap region, the fixed loop and the tucked tail may be formed from a single, continuous suture. The tucked tail comprises a first free end of the single, continuous suture that is spliced into a second free end of the single, continuous suture. The locking point comprises a locking splice.

In another embodiment, a “closed” knotless suture tissue connecting construct comprises: a suture, the suture including a finger trap region, a fixed loop and a tucked tail, the finger trap region including a lumen, a first end and a second end, the lumen extends between the first and second end, the fixed loop extending outwardly from the first end of the finger trap region, the fixed loop having a locking point and a loop opening, the tucked tail passes through the loop opening of the fixed loop and is inserted into the second end of the lumen of the finger trap region to exit out of the first end of the lumen of the finger trap region, a free end of the tucked tail is movable relative to the finger trap region from a first position to a second position, the second position extending distally from the finger trap region.

In another embodiment, a method of making a closed knotless suture construct comprising the steps of: forming a fixed loop having a fixed point and a fixed loop opening with a suture; tucking a first free end of the suture into a second free end of the suture to create a single, tucked tail with a tucked tail free end; passing the tucked tail through the fixed loop opening of the fixed loop; piercing a portion of the tucked tail with a tucked tail free end at a first location and extending towards a second location; and extending the tucked tail free end out of the second location to create a finger trap region, the finger trap region extending between the first and the second location. Furthermore, the method of making a closed knotless suture construct further comprises the step of pulling the tucked tail free end to enable the tucked tail to be movable from a first position proximal to the first end of the finger trap region, to a second position distal to the first end of the finger trap region.

### BRIEF DESCRIPTION OF THE DRAWINGS

FIG. 1 shows a plan view of a first embodiment of an open tissue connecting construct;

FIG. 2 shows a first step in the deployment of the construct of FIG. 1;

FIG. 3 shows a second step in the deployment of the construct of FIG. 2;

FIG. 4 shows a third step in the deployment of the construct of FIG. 1;

FIG. 5 shows a plan view of a second embodiment of an open tissue connecting construct;

FIG. 6 shows a first step in the deployment of the construct of FIG. 5;

FIG. 7 shows a second step in the deployment of the construct of FIG. 5;

FIG. 8 shows a third step in the deployment of the construct of FIG. 5;

FIGS. 9A-9B depict a first embodiment of a closed tissue connecting suture locking construct;

FIG. 10A depicts a side view of a second embodiment a closed tissue connecting suture locking construct;

FIG. 10B depicts a side view of a third embodiment of a closed tissue connecting construct;

FIG. 11 depicts a side view of a fourth embodiment of a closed tissue connecting suture locking construct;

FIGS. 12A-12D shows one embodiment of a method of assembly of the closed tissue connecting suture locking construct of FIGS. 9A-9B;

FIG. 13 shows a flowchart of the method of assembly of the closed tissue connecting suture locking construct of FIGS. 12A-12D;

FIG. 14 shows a flowchart of the method of assembly of the closed tissue connecting suture locking construct of FIG. 10A; and

FIG. 15 shows a flowchart of the method of assembly of the closed tissue connecting suture locking construct of FIGS. 10B and 11.

### DETAILED DESCRIPTION AND EMBODIMENTS

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter.

Numerous specific details are set forth in the following description in order to provide a thorough understanding of the invention. However, the invention may be practiced according to the claims without some or all of these specific details. Furthermore, these embodiments are described with sufficient detail to enable those skilled in the art to practice the invention, and it is to be understood that other embodiments may be employed, and that structural and logical changes may be made without departing from the spirit or scope of the present invention.

The embodiments herein are directed to a continuous loop or closed loop suture construct and its assemblies, methods of manufacturing the same and/or methods of use. All continuous or closed suture construct assemblies may comprise biocompatible materials for use as a surgical implant, and are thus preferably sterilized and packaged accordingly.

Continuous loop suture and button assemblies are commonly used during orthopedic surgery for bone-soft tissue, bone-to-bone and/or soft tissue-to-soft tissue, such as during cruciate reconstruction and/or meniscal repair. For exemplary purposes only, the embodiments will be described below with reference to an arthroscopic meniscal knee joint repair. However, the invention is not limited to this exemplary embodiment and has applicability to any surgical repair that involve reattachment of soft tissue-to-soft tissue (for example, reattachment of labral tissue (cartilage) to the glenoid), bone-to-bone and/or bone-to-soft tissue (e.g., ACL reconstruction).

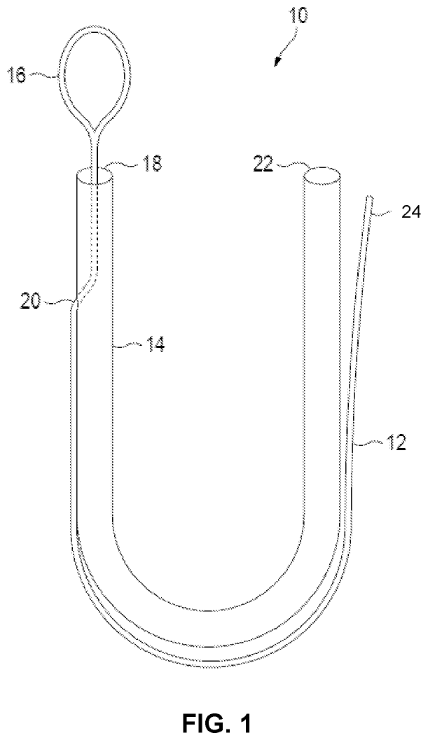

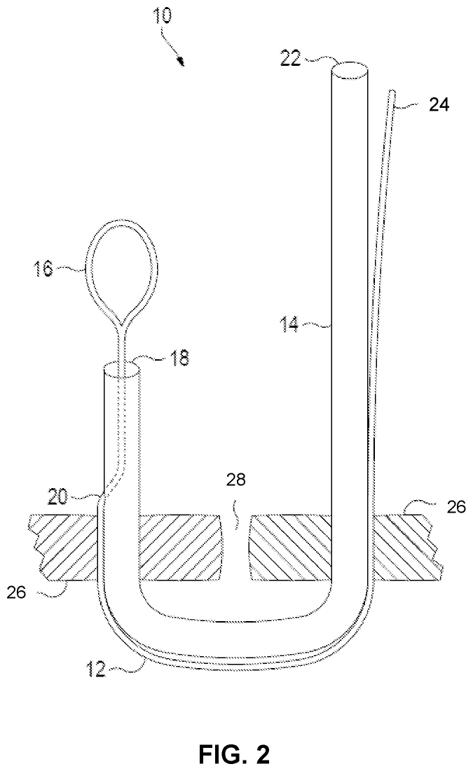

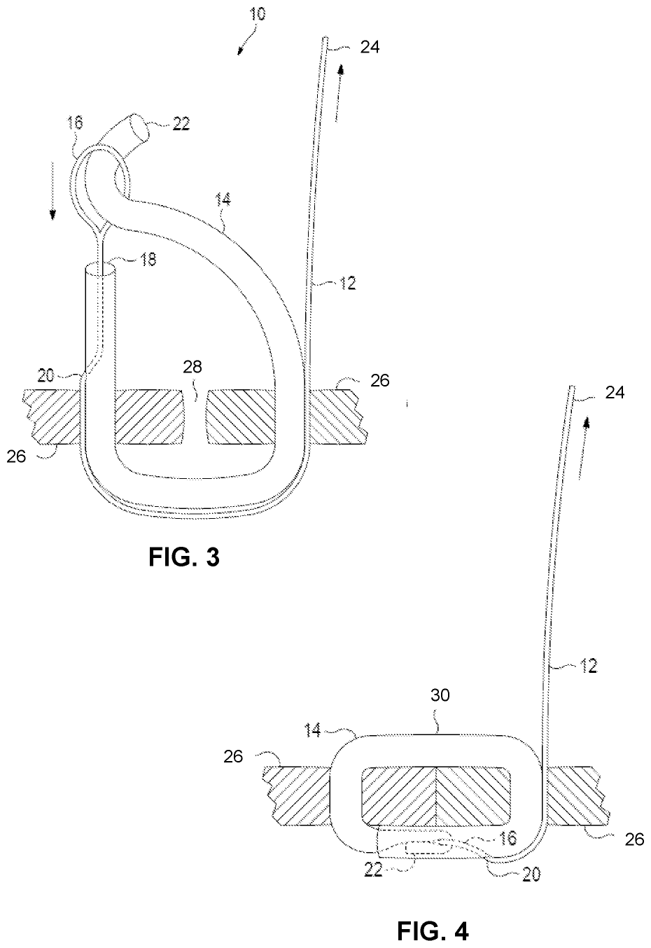

Referring to FIG. 1, an “open” tissue connecting construct 10 includes a shuttle 12 engaged to a suture 14. Shuttle 12 includes a loop 16 and a pull end 24. Shuttle 12 enters the lumen of suture 14 at a stiffened opening 18, broaches strand 14 at broach point 20, where shuttle 12 exits strand 14. Strand 14 has a transitioned end 22, which is thinner than most of strand 14, and may be achieved by splicing a thinner suture onto the thicker suture that forms the bulk of strand 14. FIGS. 2-4 show the use of construct 10, with FIG. 2 showing construct 20 engaged to two tissue extents 26, with shuttle 12 and strand 14 both passing through the tissue extents 30. Tissue extents 26 may be, for example, a portion of meniscus with a tear 28 separating the two extents 26. In FIG. 3, the transitioned end 22 of repair strand 14 is engaged to loop 16 of shuttle 12. The pull end of shuttle 12 is then pulled, causing end 22 to be pulled into stiffened opening 18. FIG. 4 shows the final result, after the pull end 24 is further pulled, pulling a portion of strand 14 in the lumen of another portion of strand 14, thereby forming a loop 30. In one method, loop 16 is left inside of stiffened opening 16, which has been pulled to the underside of tissue extents 26, which are pulled together, repairing tear 28. A finger trap is formed, negating any need to tie a knot, and forming a tight bond holding loop 30 and thereby tissue extents 26 together.

Notably, the performance of the method is greatly facilitated by the stiffened opening 18, which far more easily accepts end 22. This acceptance is also facilitated by the reduced size of transitioned end 22. In some embodiments the transition end 22 of the repair strand 14 is joined to the pull end 24 of the shuttle 12 to facilitate passing these two ends 24 and 22 through tissue extents 26. In various embodiments this is achieved by stitching the two ends 24 and 22 together, passing one through the other or splicing one into the other. In most methods, a needle is attached to the combined end of 24 and 22 to pierce through tissue extents 26 and pull ends 24 and 22 through. After successfully passing ends 24 and 22 through tissue extents 26, ends 24 and 22 can be separated, as shown in FIG. 3.

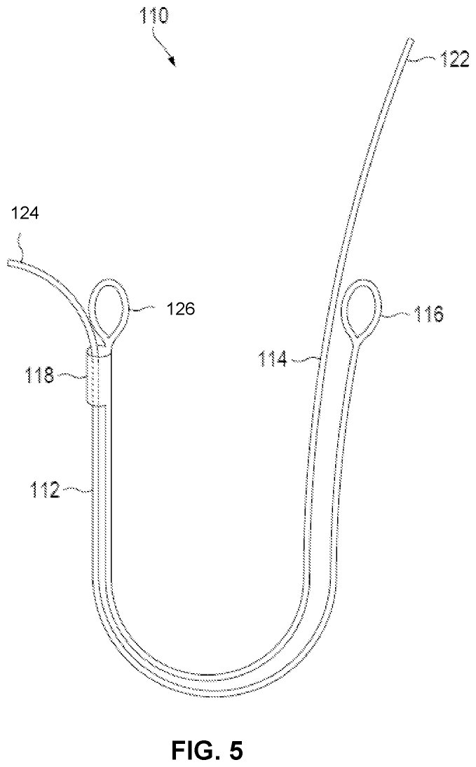

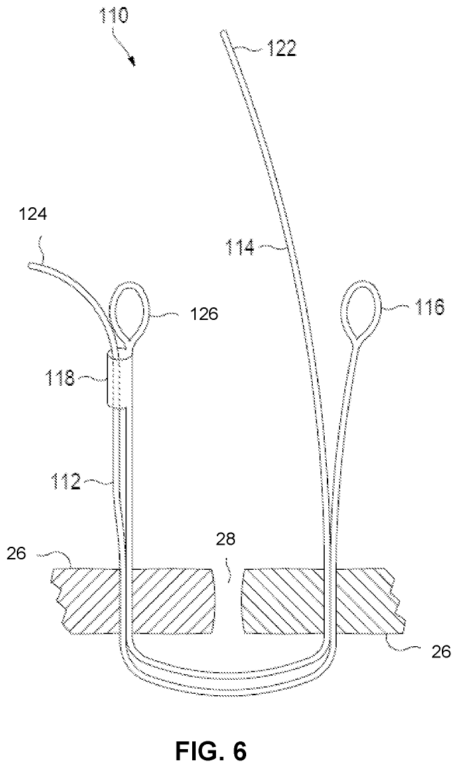

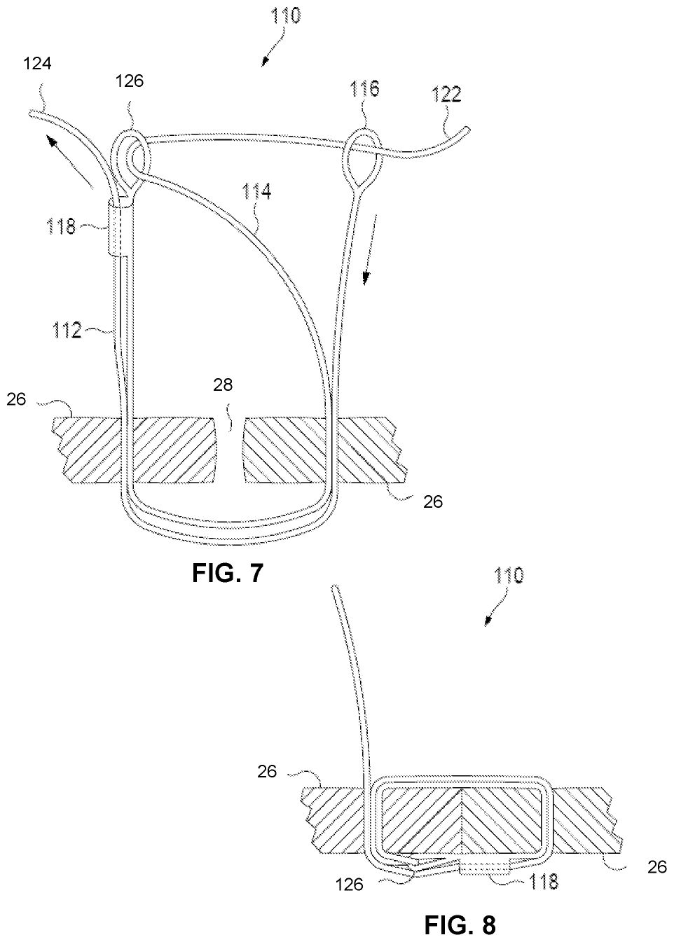

Referring to FIG. 5, in an alternative embodiment of a tissue connection construct 110, the position of the shuttle 112 is reversed, with the shuttle loop 116 being displaced from the stiffened opening 118 and with the pull end 124 emerging from the stiffened opening 118 of the repair strand 114. Also, a repair strand 114 includes a fixed loop 126, affixed to stiffened opening 118. Referring to FIGS. 6-8, to deploy, the loop 116 and the transitioned end 122 are pushed through both tissue extents 26, in one embodiment by attaching both loop 116 and end 122 to a needle that is used to pierce tissue extents 26. Transitioned end 122 is threaded first through fixed loop 130 and then through shuttle loop 116 and pull end 117 is pulled to pull first the shuttle loop 116 through both tissue extents 26, and the fixed loop 130 through the right most tissue extent 26 (as it appears in FIGS. 6-8). In FIG. 8, shuttle 112 has been entirely pulled free and is not shown. Similar to the method described in conjunction with FIGS. 2-4, shuttle loop 124 is in some embodiments joined to transitioned end 122 to facilitate passage through tissue extents 26. The benefits of stiffened opening 118 and transitioned end 122 are also evident in this method.

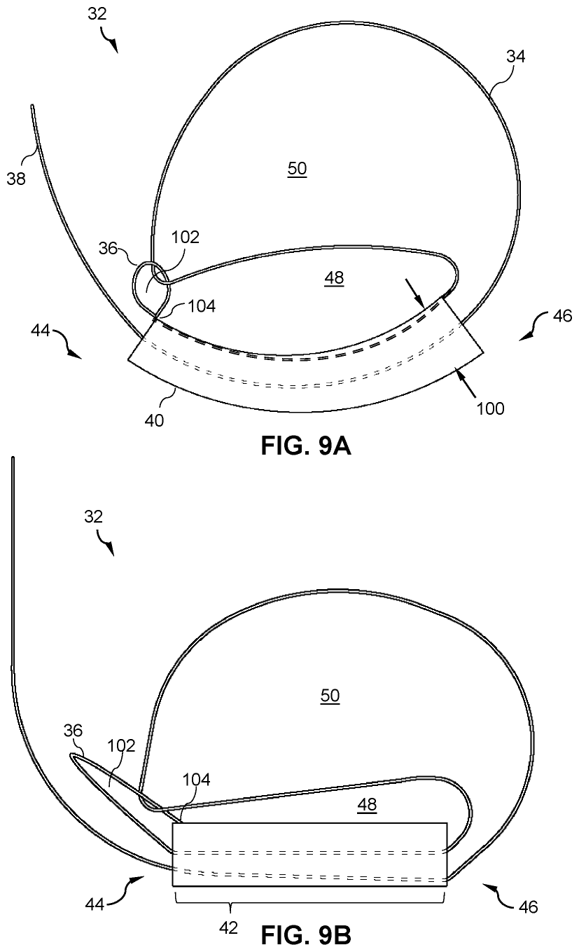

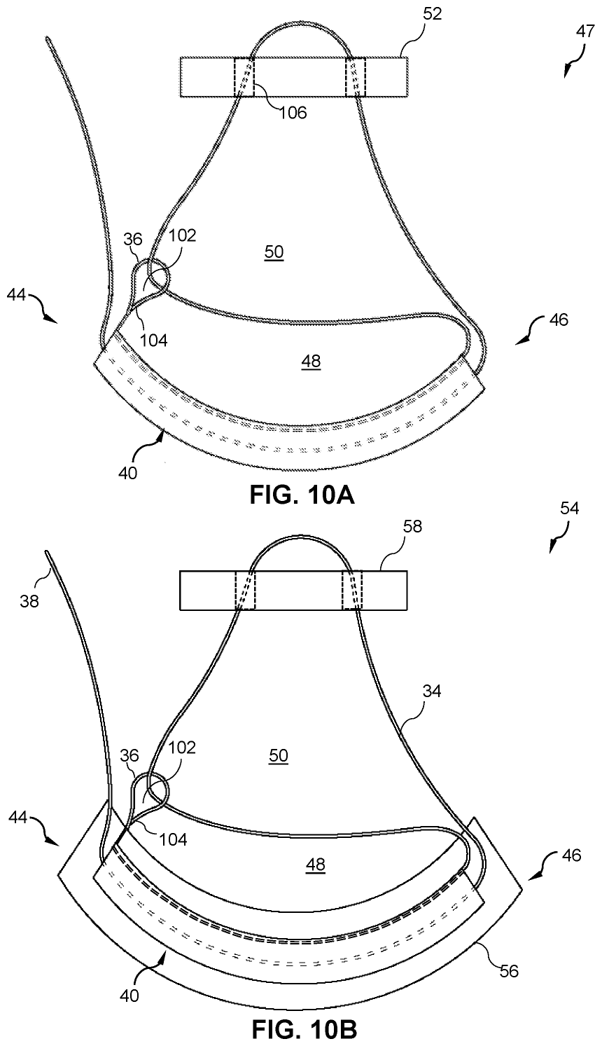

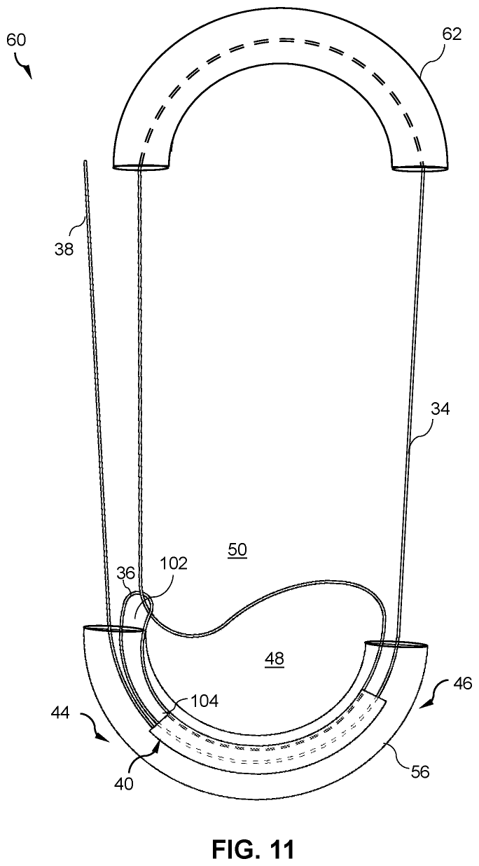

FIGS. 9A-9B, 10A-10B, 11 and 12A-12D depict several embodiments of a “closed” or “continuous” knotless suture tissue connecting locking construct 32, 47, 54, 60. The “closed” or “continuous” knotless suture tissue connecting locking construct 32, 47, 54, 60 comprises a fixed loop 36, a tucked tail free end 38 or a free pull tail end 38, a finger trap region 40 and one or more sliding suture loops 48,50 (see FIGS. 9A-9B). The “closed” or “continuous” knotless suture tissue connecting locking construct 32, 47, 54, 60 may further comprise a first anchoring construct 52 (see FIG. 10A). The “closed” or “continuous” knotless suture tissue connecting locking construct 32, 47, 54, 60 may further comprise a first anchoring construct 52 and a second anchoring construct 56 (see FIGS. 10B and 11). The “closed” suture anchor construct assemblies 32, 47, 54, 60 having one or more anchoring constructs enclosed by a continuous (i.e., closed) loop of a suture or repair strand are also disclosed herein. Preferred assemblies closed suture anchor constructs are constructed such that the anchoring constructs cannot be detached from the continuous or closed loop suture anchor construct 32, 47, 54, 60 without breaking or opening the continuous loop of the closed suture anchor construct 32, 47, 54, 60.

FIGS. 9A-9B depict a first embodiment of a “closed” or “continuous” knotless suture tissue connecting locking construct 32, 47, 54, 60. The “closed” or “continuous” knotless suture tissue construct 32, 47, 54, 60 comprises a finger trap region 40, a fixed loop 36, a tucked tail free end 38. In one embodiment, “closed” or “continuous” knotless suture tissue construct 32, 47, 54, 60 comprises a suture 34, the suture including a finger trap region 40, a fixed loop 36 and a tucked tail free end 38, the finger trap region 40 including a lumen (not shown), a first end 44 and a second end 46, the lumen extends between the first end 44 and second end 46. The fixed loop 40 extending outwardly from the first end 44 of the finger trap region 40, the fixed loop 36 having a locking point 104 and a loop opening 102, the tucked tail 38 passes through the loop opening 102 of the fixed loop 36 and is inserted into the second end 46 of the lumen of the finger trap region 40 to exit out of the first end 44 of the lumen of the finger trap region 40, a free end of the tucked tail 38 is movable relative to the finger trap region 40 from a first position to a second position, the second position extending distally from the finger trap region 40.

Alternatively, in another embodiment, the “closed” knotless suture tissue connecting construct comprises: a suture 34, the suture 34 including a finger trap region 40, a fixed loop 36 and a tucked tail free end 38, the finger trap region 40 including a lumen, a first end 44 and a second end 46, the lumen extends between the first end 44 and second end 46. The fixed loop 36 extending outwardly from the first end 44 of the finger trap region 40, the fixed loop 36 having a locking point 104 and a loop opening 102, the tucked tail 38 passes through the loop opening 102 of the fixed loop 36 and is inserted into the second end 46 of the lumen of the finger trap region 40 to exit out of the first end 44 of the lumen of the finger trap region 40, a free end of the tucked tail 38 is movable relative to the finger trap region 40 from a first position to a second position, the second position extending distally from the first end 44 of the finger trap region 40.

The finger trap region 40 including a first end 44 and a second end 46. The finger trap region 40 may further comprise a lumen. The finger trap region 40 may further comprise a length 42 and a width 100. The finger trap length 42 may comprise at least 5 mm or greater, at least 7 mm or greater, and/or at least 10 mm or greater. The finger trap width 100 may comprise a width at least 1.5 times greater or at least 2 times greater than a diameter of the tucked tail free end 38 or the diameter of the suture 34.

The fixed loop 36 may comprise a fixed loop opening 102 and a locking point 104. The fixed loop 36 extending outwardly from the first end 44 of the finger trap region 40. The locking point 104 may comprise a locking splice or a knot. The locking splice may include a brummel splice (double, triple or quadruple), eye splice, chain splice, back splice, a grog sliding splice and/or any combination thereof. The locking point 104 of the fixed loop 36 is intended to fix the loop opening 102 and prevent the loop opening 102 from changing in loop opening size or diameter.

The tucked tail 38 passes through the fixed loop opening 102 of the fixed loop 36. Accordingly, the tucked tail 38 passes through the fixed loop opening 102 to create one or more sliding loops 48, 50. The tucked tail 38 is inserted or pierced into the second end 46 of the finger trap region 40. At least a portion of the tucked tail 38 extends along the length 52 of the finger trap region 40 to exit out of the first end 44 of the finger trap region 40. Accordingly, at least a portion of the tucked tail 38 extends along the length 52 of the finger trap region 40 to exit out of the first end 44 of the finger trap region 40 to create one or more sliding loops 48,50. The tucked tail free end 38 is movable relative to the finger trap region 40 from a first position to a second position, the second position extending distally from the first end of the finger trap region 40. Alternatively, the tucked tail free end 38 is movable relative to the finger trap region 40 from a first position proximal to the first end of the finger trap region, to a second position distal to the first end of the finger trap region. The tucked tail 38 may comprise a first free end of a suture that is inserted into a second free end of a suture to form a single, tucked tail free end. The suture 34 may comprise a repair strand.

The finger trap region 40, the fixed loop 36 and the tucked tail free end 38 may be formed from a single, continuous suture or a plurality of sutures. In one embodiment, the tucked tail 38 comprises a first free end of the single, continuous suture that is spliced into a second free end of a suture or repair strand. The suture or repair strand 34 may comprise a flat suture, a round suture or a flat-to-round suture. The suture may comprise a multifilament, braided suture. The suture may comprise a material. The material may include absorbable or non-absorbable materials. The material may comprise synthetic or natural suture materials. The suture material may further comprise a ultra-high weight molecular polyethylene (UHWMPE), polyethylene (PE), nylon, polyethylene terephthalate (PET), and/or any combination thereof.

FIG. 10A depicts a second embodiment of a “closed” or “continuous” knotless suture tissue connecting locking construct 32, 47, 54, 60. The “closed” or “continuous” knotless suture tissue connecting locking construct 32, 47, 54, 60 further comprises at least one anchoring construct 52, 56. The at least one anchoring construct 52, 56, 62 may comprise a soft, flexible, textile anchoring construct or a hard anchoring construct. In one embodiment, a “closed” or “continuous” knotless suture tissue connecting locking construct 32, 47, 54, 60 comprises at least one anchoring construct 52, 56, 62, the at least one anchoring construct 52, 56, 62 may comprise a suture button or a button 52. The at least one anchoring construct 52, 56, 62 may comprise an anchor 56, 62, the anchor 56, 62 may comprise a textile suture anchor or a hard anchor.

The tucked tail 38 engages with the button 52. The tucked tail 38 is threaded or inserted through at least two apertures 106 disposed within the button 52. The button 52 may comprise a material, the material may include a polymer, ceramic or a metal. The material may further comprise a biocompatible material. The metal may include steel, titanium, cobalt, steel alloys, titanium alloys and/or cobalt alloys. The polymer may include a thermoset or thermoplastic polymer. Any suitable number and shapes of apertures 106 can be disposed within the button 52. Aperture shape can be circular, ovular, square, and/or rectangular. Non-limiting examples of buttons 52 that can be used with the teachings herein, include the button 52 of the XO BUTTON® commercially available by CONMED®/LINVATE® and suitable buttons disclosed in U.S. Pat. No. 6,533,802 to Bojarski et al., and U.S. Pat. No. 5,306,301 to Graf et al. Furthermore, the button 52 may comprise a shape, the shape include oval, racetrack, circular, square, rectangular.

FIGS. 10B and 11 depict a third and fourth embodiment of a “closed” or “continuous” knotless suture locking construct 32, 47, 54, 60. The “closed” or “continuous” knotless suture locking construct 32, 47, 54, 60 further comprises a plurality of anchoring constructs 52, 56, 62, and/or a first anchoring construct and a second anchoring construct. The plurality anchoring constructs 52, 56, 62, the first anchoring construct and the second anchoring construct may comprise a soft, flexible textile anchoring construct or a hard anchoring construct. In one exemplary embodiment, a “closed” or “continuous” knotless suture tissue connecting locking construct 32, 47, 54, 60 comprises a first anchoring construct and a second anchoring construct. The first anchoring construct may comprise a suture button or a button 52 or an anchor 56, 62. The second anchoring construct may comprise a suture button or a button 52 or an anchor 56, 62. The anchor 56, 62 may comprise a textile suture anchor or a hard anchor.

In another embodiment, the “closed” or “continuous” knotless suture locking construct 32, 47, 54, 60 comprises: a first anchoring construct, a second anchoring construct, and a suture 34, the suture 34 including a finger trap region 40, a fixed loop 36 and a tucked tail free end 38, the finger trap region 40 including a lumen, a first end 44 and a second end 46, the lumen extends between the first end 44 and second end 46. The fixed loop 36 extending outwardly from the first end 44 of the finger trap region 40 and a portion of the fixed loop 36 extending outside a sidewall of the first anchoring construct 56 (see FIG. 10B) and/or a portion extending outside a first end of the lumen of the first anchoring construct 56 (see FIG. 11), the fixed loop 36 having a locking point 104 and a loop opening 102, the tucked tail 38 passes through the loop opening 102 of the fixed loop 36 and engages with the second anchoring construct 52 (see FIG. 10B) and/or extends through the lumen of the second anchoring construct 62 (see FIG. 11). The tucked tail 38 is inserted into the second end 46 of the lumen of the finger trap region 40 and/or inserted into a second end of the first anchoring construct 56 to exit out of the first end 44 of the lumen of the finger trap region 40 and/or exit out of the first end of the lumen of the first anchoring construct 56, a free end of the tucked tail 38 is movable relative to the finger trap region 40 from a first position to a second position, the second position extending distally from the first end 44 of the finger trap region 40. The first anchoring construct 56 may comprise a button or an anchor. The second anchoring construct 52, 62 may comprise a button or an anchor. The anchor includes a textile suture anchor or a hard anchor.

Method of Assembly of a Closed Knotless Suture Construct

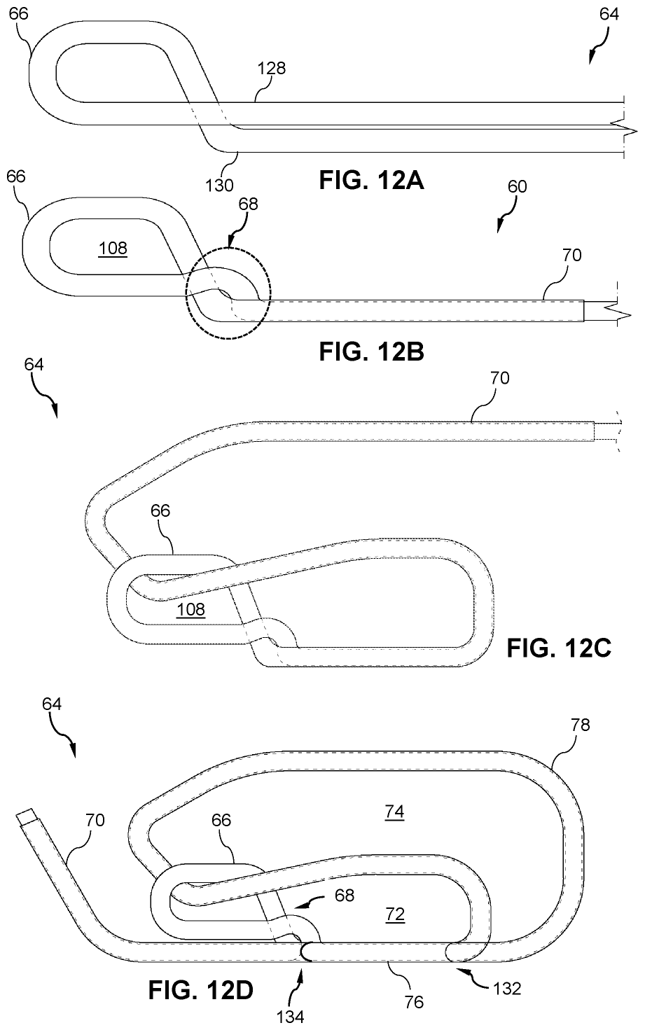

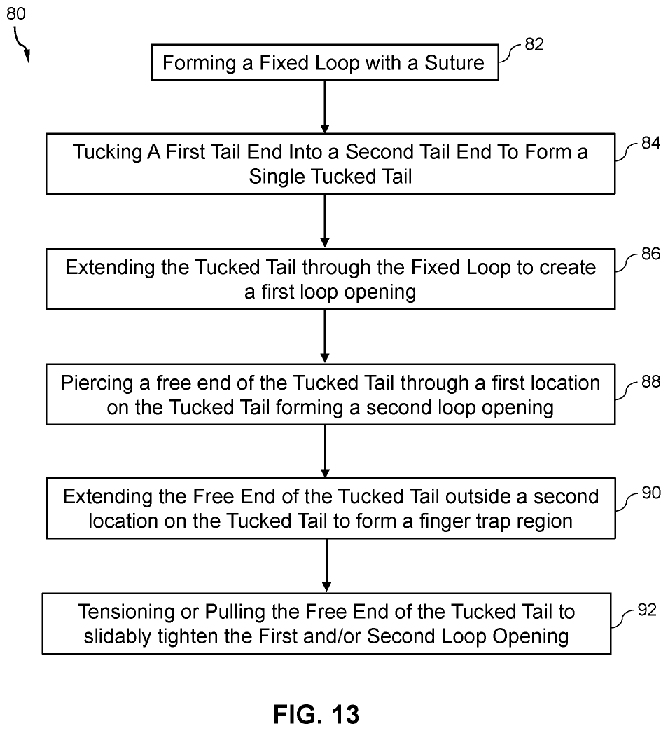

FIGS. 12A-12D and FIG. 13 illustrate one embodiment of a method to assemble 80 the closed knotless suture construct 32, 47, 54, 60 of FIGS. 9A-9B. In one embodiment, the method of assembling 80 a closed knotless suture construct 32, 47, 54, 60 comprises the steps of: Forming a fixed loop 66 having a fixed point 68 and a fixed loop opening 108 with a suture 82; tucking a first free end 128 of the suture into a second free end 130 of the suture to create a single, tucked tail free end 70, 84; passing the tucked tail free end 70 through the fixed loop opening 108 of the fixed loop 86; piercing a portion of the tucked tail with a tucked tail free end 70 at a first location 132 and extending towards a second location 134, 88; and extending the tucked tail free end 70 out of the second location 134 to create a finger trap region 76, the finger trap region 76 extending between the first location 132 and the second location 132, 90. The method of assembling further comprises obtaining a suture, the suture comprising a first free end 128 and a second free end 130. The step of passing the tucked tail 70 through the fixed loop opening 108 of the fixed loop 66 further comprises passing the tucked tail 70 through the fixed loop opening 108 of the fixed loop 66 to create a first sliding loop 72. The step of piercing a portion of the tucked tail body with a tucked tail free end 70 at a first location 132 and extending towards a second location 134 further comprises the step of piercing a portion of the tucked tail body with a tucked tail free end 70 at a first location 132 and extending towards a second location 134 to create a second sliding loop 74.

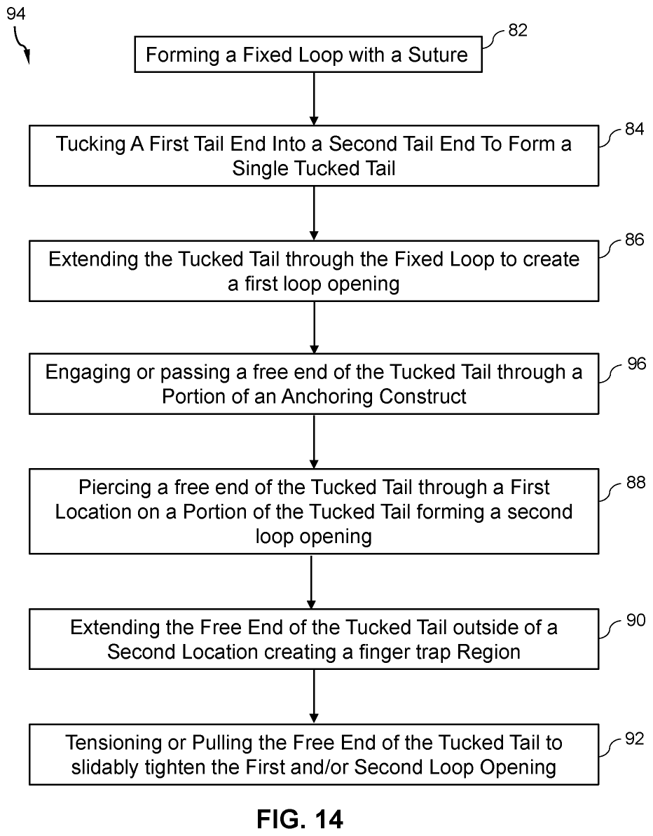

FIG. 14 illustrates a second embodiment of a method to assembly 94 the closed knotless suture anchor construct 32, 47, 54, 60. In one embodiment, the method of assembling 80 a closed knotless suture construct 32, 47, 54, 60 comprises the steps of: Forming a fixed loop having a fixed point 68 and a fixed loop opening 108 with a suture 82; tucking a first free end 128 of the suture into a second free end 130 of the suture to create a single, tucked tail free end 70, 84; passing the tucked tail free end 70 through the fixed loop opening 108 of the fixed loop 86; engaging the tucked tail 70 through a portion of at least one anchoring construct 96; piercing a portion of the tucked tail with a tucked tail free end 70 at a first location 132 and extending towards a second location 134, 88; and extending the tucked tail free end 70 out of the second location 134 to create a finger trap region 76, the finger trap region 76 extending between the first location 132 and the second location 132, 90. The method of assembling further comprises obtaining a suture, the suture comprising a first free end 128 and a second free end 130.

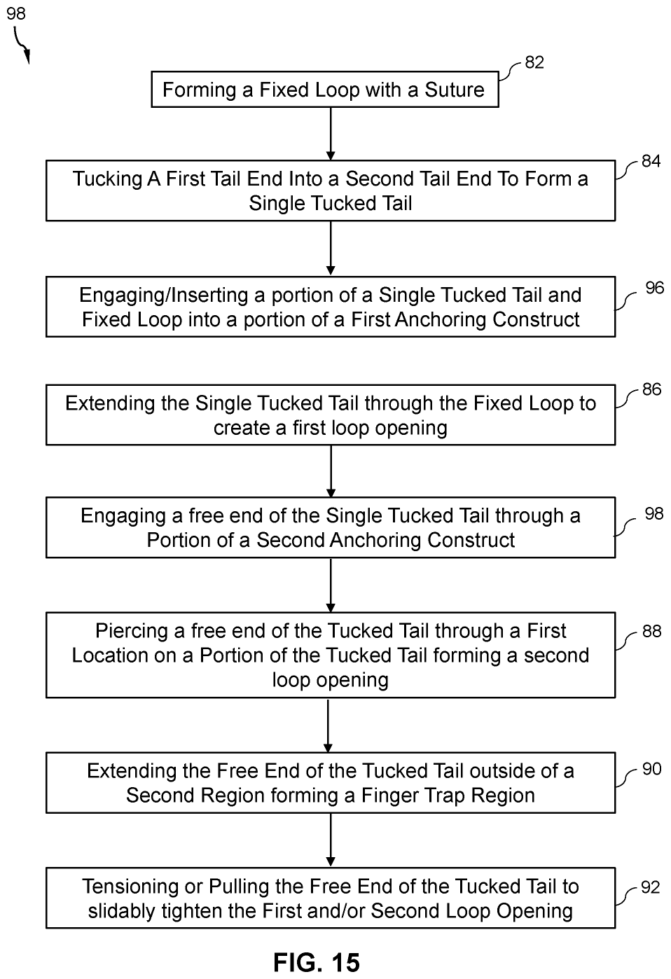

FIG. 15 illustrates a third embodiment of a method to assembly 98 the closed knotless suture anchor construct 32, 47, 54, 60. In one embodiment, the method of assembling 80 a closed knotless suture construct 32, 47, 54, 60 comprises the steps of: Forming a fixed loop 66 having a fixed point 68 and a fixed loop opening 108 with a suture 82; tucking a first free end 128 of the suture into a second free end 130 of the suture to create a single, tucked tail free end 70, 84; engaging or inserting at least a portion of the tucked tail 70, fixed loop 66 and a finger trap region 76 through a portion of first anchoring construct 96; engaging or extending the tucked tail free end 70 through a second anchoring construct 98; passing the tucked tail free end 70 through the fixed loop opening 108 of the fixed loop 86; piercing a portion of the tucked tail with a tucked tail free end 70 at a first location 132 and extending towards a second location 134, 88; and extending the tucked tail free end 70 out of the second location 134 to create a finger trap region 76, the finger trap region 76 extending between the first location 132 and the second location 132, 90.

The method of assembling 80, 94, 98 further comprises obtaining a suture, the suture comprising a first free end 128 and a second free end 130. The method of assembling 80, 94, 98 further comprises the step tensioning or pulling the tucked tail 70 upwardly to tighten the first 72 and/or second loop opening 74. The step of passing the tucked tail 70 through the fixed loop opening 108 of the fixed loop 66 further comprises passing the tucked tail 70 through the fixed loop opening 108 of the fixed loop 66 to create a first sliding loop 72. The step of piercing a portion of the tucked tail body with a tucked tail free end 70 at a first location 132 and extending towards a second location 134 further comprises the step of piercing a portion of the tucked tail body with a tucked tail free end 70 at a first location 132 and extending towards a second location 134 to create a second sliding loop 74.

Accordingly, the suture is formed a single, continuous suture. The single, continuous suture comprises a round or flat suture. The locking point comprises a locking splice. The locking splice comprises a brummel splice. The first anchoring construct comprises a first button or a first anchor, and the second anchoring construct comprises a second button or second anchor. The first or second anchor comprises a textile suture anchor or a hard anchor.

Method of Use

In use, soft tissues can be pierced by and/or captured in the repair loop, with a tension subsequently drawn on the adjustment suture, which desirably tightens the repair loop and trap and “locks” the construct closed in a desired position in a knotless locking arrangement.

Alternatively, the method of use may comprise the steps of: obtaining a closed suture construct, the closed suture construct comprising: a first anchoring construct, a second anchoring construct, and a suture 34, the suture 34 including a finger trap region 40, a fixed loop 36 and a tucked tail free end 38, the finger trap region 40 including a lumen, a first end 44 and a second end 46, the lumen extends between the first end 44 and second end 46. The fixed loop 36 extending outwardly from the first end 44 of the finger trap region 40 and a portion of the fixed loop 36 extending outside a sidewall of the first anchoring construct 52 (see FIG. 10B) and/or outside a first end of the lumen of the first anchoring construct 52 (see FIG. 11), the fixed loop 36 having a locking point 104 and a loop opening 102, the tucked tail 38 passes through the loop opening 102 of the fixed loop 36 and engages with the second anchoring construct (see FIG. 10B) and/or extends through the lumen of the second anchoring construct (see FIG. 11). The tucked tail 38 is inserted into the second end 46 of the lumen of the finger trap region 40 to exit out of the first end 44 of the lumen of the finger trap region 40, a free end of the tucked tail 38 is movable relative to the finger trap region 40 from a first position to a second position, the second position extending distally from the first end 44 of the finger trap region 40; deploying a first anchoring construct of the closed suture construct onto a first surface of a tissue; retracting the closed suture construct until reaching a second surface of the tissue; deploying a second anchoring construct of the closed suture construct onto the second surface of the tissue; tensioning the tucked tail 38 to enable the movement relative to the finger trap region 40 to set the first and second anchoring construct on the first surface and second surface of the tissue.

The method of use further comprises inserting the closed suture construct inferior to the tear to deploy the first anchoring construct. The method of use further comprises inserting the closed suture construct inferior to the tear to deploy the first anchoring construct and retracting the closed suture construct superior to the tear to deploy the second anchoring construct. The method of use further comprises the step of confirming the setting of the first and second anchoring construct on the first surface and second surface of the tissue by the stopping or inhibition of movement.

The first surface may comprise an anterior surface of the meniscus and the second surface is the posterior surface of the meniscus. The first surface may comprise an anterior surface of a bone and the second surface is the posterior surface of the meniscus. Alternatively, the first surface may comprise an anterior surface of the meniscus and inferior to the tear, and the second surface is the anterior surface of the meniscus and superior to the tear. The first surface may comprise an anterior surface of a bone and the second surface is the posterior surface of the meniscus.

In various embodiments, the locking construct could be employed in a fully assembled and configured state for a variety of surgical procedures, or the locking construct could be partially assembled (i.e., such as where the adjustable suture has not yet been inserted through the lumen and/or the loop in not fully formed around the anchor), where a shuttle loop could be employed to draw the repair suture or strand back through the trap (e.g., after the physician manipulates the repair suture to achieve a variety of objectives) to effectuate creation of a repair loop around the tissue(s) desiring to be approximated, with the desired knotless locking actions being completed after tissue approximation is completed.

In various exemplary embodiment, the disclosed knotless locking construct could be incorporated into an anchorless surgical device, which might have particular utility as a tissue connecting construct utilized for tissue-to-tissue connections, including capsule closure procedures in the hip, syndesmosis repairs and/or other similar procedures. In other embodiments, the disclosed knotless locking construct could be employed with a single anchor (including both hard and/or soft anchors), wherein the locking construct might be fully contained within the anchor, partially contained within the anchor and/or may be positioned fully outside of the anchoring device. Such a locking construct could be widely used with a variety of anchor designs (e.g., all-suture, PEEK, Titanium, etc.) in a wide variety of surgical procedures, including soft tissue to bone approximation (e.g., ACL repair procedures). In still other embodiments, the disclosed knotless locking construct might be employed with a plurality of anchors and/or anchor buttons or similar devices, with the locking construct possibly fully contained within one of the anchor, partially contained within one of the anchors and/or may be positioned fully outside of the anchoring devices. Such a locking construct could be utilized in a wide variety of surgical procedures, including bone to bone approximation (e.g., Syndesmosis procedures, etc.).

The anchor construct may optionally incorporate one or more larger anchor components to accommodate the locking construct therein as compared to traditional anchor designs, which could increase the complexity, adjustability and/or assembly difficulty of the system to a limited extent. However, the increased construct strength (e.g., using a 2-way bowtie design) and larger usable space (providing more adjustability) granted to a surgeon by this embodiment should heavily outweigh any potential negative factors.

In various alternative embodiments, suture tapes may similarly be incorporated into various components of the disclosed suture anchor constructs.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure. While embodiments and applications of the present subject matter have been shown and described, it should be apparent to those skilled in the art that many more modifications are possible without departing from the inventive concepts herein. The subject matter, therefore, is not to be restricted except in the spirit of the appended claims. Thus, while embodiments and applications of the present subject matter have been shown and described, it should be apparent that other embodiments, applications and aspects are possible and are thus contemplated and are within the scope of this application.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What I/we claim:

1. A “closed” knotless suture tissue connecting construct comprises:
A suture, the suture including a finger trap region, a fixed loop and a tucked tail, the finger trap region including a lumen, a first end and a second end, the lumen extends between the first and second end, the fixed loop extending outwardly from the first end of the finger trap region, the fixed loop having a locking point and a loop opening, the tucked tail passes through the loop opening of the fixed loop and is inserted into the second end of the lumen of the finger trap region to exit out of the first end of the lumen of the finger trap region, a free end of the tucked tail is movable relative to the finger trap region from a first position to a second position, the second position extending distally from the first end of the finger trap region.

2. The closed knotless suture tissue connecting construct of claim 1, wherein the suture is formed from a single, continuous suture.

3. The closed knotless suture tissue connecting construct of claim 2, wherein the single, continuous suture comprises a round or flat suture.

4. The closed knotless suture tissue connecting construct of claim 1, wherein the locking point comprises a locking splice.

5. The closed knotless suture tissue connecting construct of claim 4, wherein the locking splice is a brummel splice.

6. The closed knotless suture tissue connecting construct of claim 1, wherein the tucked tail comprises a first free end of the suture that is spliced into a second free end of the suture.

7. The closed knotless suture tissue connecting construct of claim 2, wherein the tucked tail comprises a first free end of the single, continuous suture that is spliced into a second free end of the single, continuous suture.

8. The closed knotless suture tissue connecting construct of claim 1, wherein the closed knotless suture tissue connecting construct further comprises at least one anchoring construct.

9. The closed knotless suture tissue connecting construct of claim 1, wherein the closed knotless suture tissue connecting construct further comprises a first anchoring construct and a second anchoring construct.

10. The closed knotless suture tissue connecting construct of claim 9, wherein the first anchoring construct comprises a first anchor and the second anchoring construct comprises a second anchor.

11. The closed knotless suture tissue connecting construct of claim 9, wherein the first anchoring construct comprises a suture button and the second anchoring construct comprises an anchor.

12. A method of making a closed knotless suture tissue connecting construct comprising the steps of:
Forming a fixed loop having a locking point and a fixed loop opening with a suture;
Tucking a first free end of the suture into a second free end of the suture to create a single, tucked tail with a tucked tail free end;
Passing the tucked tail through the fixed loop opening of the fixed loop;
Piercing a portion of the tucked tail with a tucked tail free end at a first location and extending towards a second location; and
Extending the tucked tail free end out of the second location to create a finger trap region, the finger trap region extending between the first and the second location.

13. The method of claim 12, wherein the suture is formed a single, continuous suture.

14. The method of claim 13, wherein the single, continuous suture comprises a round or flat suture.

15. The method of claim 12, wherein the locking point comprises a locking splice.

16. The method of claim 15, wherein the locking splice is a brummel splice.

17. The method of claim 12, wherein the method of making the closed knotless suture tissue connecting construct further comprises a step of engaging with a first anchoring construct and a second anchoring construct.

18. The method of claim 17, wherein the first anchoring construct comprises a button and the second anchoring construct comprises an anchor.

19. The method of claim 17, wherein the first anchoring construct comprises a first anchor and the second anchoring construct comprises a second anchor.

20. The method of claim 18, wherein the anchor comprises a textile suture anchor or a hard anchor.
